(19) United States
(12) Patent Application Publication
(10) Pub. No.: US 2005/0172974 A1
(43) Pub. Date: Aug. 11, 2005

(54) KufBag, restraint device for transporting prisoners

(76) Inventors: John Thomas Foster, Westerville, OH (US); Glenda Louise Hill-Foster, Westerville, OH (US)

Correspondence Address: John T. Foster, 1333 Cobblestone Ave., Westerville, OH, 43081, US

(21) Appl. No.: 10/952,586

(22) Filed: Sep. 26, 2004

Related U.S. Application Data
(60) Provisional application No. 60/543,185, filed on Feb. 10, 2004.

(51) Int. Cl. A61F 5/37

(52) U.S. Cl. 128/878

(57) ABSTRACT

The KufBag secures the hands together to eliminate grasping while departmentalizing the hands to keep them separated. The KufBag has grommet holes to permit the use of plastics ties. The KufBag has a velcro alarm system that makes noise when the prisoner tries to escape. The KufBag has clip buckle for added safety to preclude escaping.

### CROSS-REFERENCE TO RELATED APPLICATIONS

Provisional Patent Application, No. 60/543,185, Filing Date Feb. 10, 2004, Confirmation No. 1580

### FIELD OF INVENTION

KufBag is a restraint system utilized in the transporting of a prisoner from Point A. To B. and even to Point C. and D. and beyond. We provide protection and safety for the person transporting and to the prisoner that is being transported. The securing of hands for transporting a prisoner prevents the use of hands, which can and has caused harm because of the cuffed hands still being free. The KufBag offers humanity towards the apprehended, removing cause of impulsive. It will also decrease attacks against such prisoners following arrest or during transport.

### STATEMENT REGARDING FEDERALLY SPONSORED RESEARCH OR DEVELOPMENT

Not applicable

### REFERENCE TO SEQUENCE LISTING. A TABLE, OR A COMPUTER PROGRAM LISTING COMPACT DISC APPENDIX

Not applicable

### BACKGROUND OF INVENTION

When transporting prisoners in handcuffs and plastic cuffs, the remaining partial freedom of hands that has lead to loss of life because of the hands not being completely secured, allowing the prisoners to grab officers and their weapons, which has been used against them, the wrist chain has been used to choke the transporting officer. Without restraints being secured on a prisoner, has caused successful Escapes and loss of life.

### BRIEF DESCRIPTION OF THE SEVERAL VIEWS OF THE DRAWING

FIG. 1 SIDE VIEW

FIG. 2 FRONT VIEW

FIG. 3 BACK VIEW LEFT

FIG. 4 BACK VIEW RIGHT

FIG. 5 SUBASSEMBLY A

FIG. 6 SUBASSEMBLY B

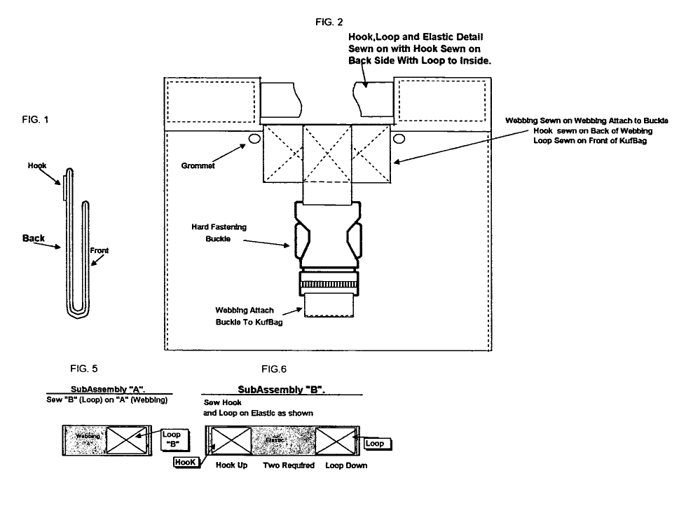

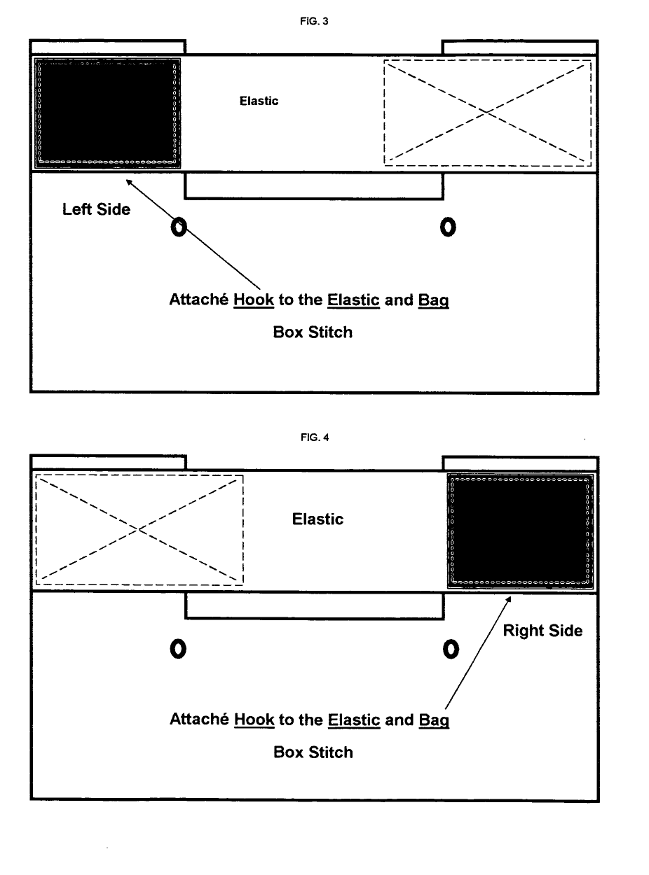

### A DETAILED DESCRIPTION OF THE INVENTION

The Kufbag is a strait jacket for the hands. The KufBag gives an added safety precaution for securing hands. The KufBag is used in conjunction with wrist straps, handcuffs, plastic ties, and a Velcro fastening clip lock mechanism. While secured in front or behind the back safety is always number one.

This product can be used by law enforcement, corrections, the military, airlines, bus lines, juvenile detention centers, hospitals, bounty hunters, and immigration officers.

The KufBag is manufactured consisting of the following materials, Nylon and Polyester Blend with foam for cushioning. Hard Plastic Fastening Buckle combined with Velcro for closing bag. Two sets of Grommets for inserting plastic ties. The KufBag is lightweight, washable and reusable.

The KufBag is unique. The difference is our product secures the hands together and eliminates grasping. By keeping the hands together yet separated a prisoner is unable to use their hands to grasp.

1. What we claim as our invention is the KufBag to be a straitjacket for the hands, the KufBag encases the hands along with the handcuffs while using plastic ties secured through grommets holes while separating or departmentalizing the hands to prevent grasping ability, to protect the transporter.

2. What we claim as our invention is the KufBag protects the transporter from bio-hazardous infection such as HIV and Hepatitis health risks, because the prisoners cannot us the hands to scratch or break surface skin of the transporter.
